(19) United States
(12) Patent Application Publication
(10) Pub. No.: US 2017/0319816 A1
(43) Pub. Date: Nov. 9, 2017

(54) SLEEP AND WAKE ENVIRONMENT APPARATUS

(71) Applicant: Enduring Wellness, LLC, Cleveland, OH (US)

(72) Inventors: Brian Sokol, Vermillion, OH (US); Anthony DeMore, Willoughby, OH (US)

(21) Appl. No.: 15/590,573

(22) Filed: May 9, 2017

Related U.S. Application Data
(60) Provisional application No. 62/333,428, filed on May 9, 2016.

(51) Int. Cl. A61M 21/02; G04B 23/02; A61M 21/00

(52) U.S. Cl. CPC A61M 21/02; G04B 23/02; A61M 2021/0005; A61M 2021/0027; A61M 2021/0016; A61M 2021/0077; A61M 2021/0044

(57) ABSTRACT

A sleep and wake environment apparatus includes an aromatherapy diffuser; a speaker; a light source; a clock; and a user interface with controls and a display. Each of these elements are in communication with a control computing device configured to control the aromatherapy diffuser, the speaker, the light source, and the clock automatically and without contemporaneous user input.

### CROSS-REFERENCE TO RELATED APPLICATIONS

This application claims priority to U.S. Provisional Patent Application No. 62/333,428, filed on May 9, 2016, and entitled “Sleep & Wake Environment Apparatus,” the entirety of which is incorporated herein by reference.

### FIELD

The present disclosure relates to sleep wellness, and more particularly to improving the sleep and wake environment.

### BACKGROUND

There are multiple products that can be purchased to create the desired sleep and wake environment. Namely, humidifiers, aromatherapy diffusers, sound therapy machines, and light therapy lamps. These products are available individually, but require a large amount of space, multiple electrical outlets, and numerous interfaces. Because these products are individual in nature, there is no opportunity for the consumer to control them from one interface to customize their sleep and wake environment.

### SUMMARY

In an embodiment a sleep and wake environment apparatus includes an aromatherapy diffuser; a speaker; a light source; a clock; and a user interface with controls and a display. Each of these elements are in communication with a control computing device configured to control the aromatherapy diffuser, the speaker, the light source, and the clock automatically and without contemporaneous user input.

In an embodiment a method of controlling a sleep and wake environment, comprising: automatically and without contemporaneous user input: activating in a pre-sleep time period at least two of an aromatherapy diffuser, a speaker, or a light source; activating, deactivating, or adjusting in a sleep time period at least one of the aromatherapy diffuser, the speaker, or the light source; and activating, deactivating, or adjusting in a wake time period at least two of the aromatherapy diffuser, the speaker, or the light source.

### BRIEF DESCRIPTION OF THE DRAWINGS

FIG. 1 shows an example sleep and wake environment apparatus.

FIG. 2 shows an example computing device for use with the sleep and wake environment apparatus.

### DETAILED DESCRIPTION

Disclosed herein is an apparatus for improving the sleep and wake environment that incorporates aromatherapy, sound therapy, light therapy, humidification, and an alarm clock. These elements are under a single control so that the end-user can customize the blend of these therapies based on their individual preferences or can use preset programs to define an ideal sleep and wake environment.

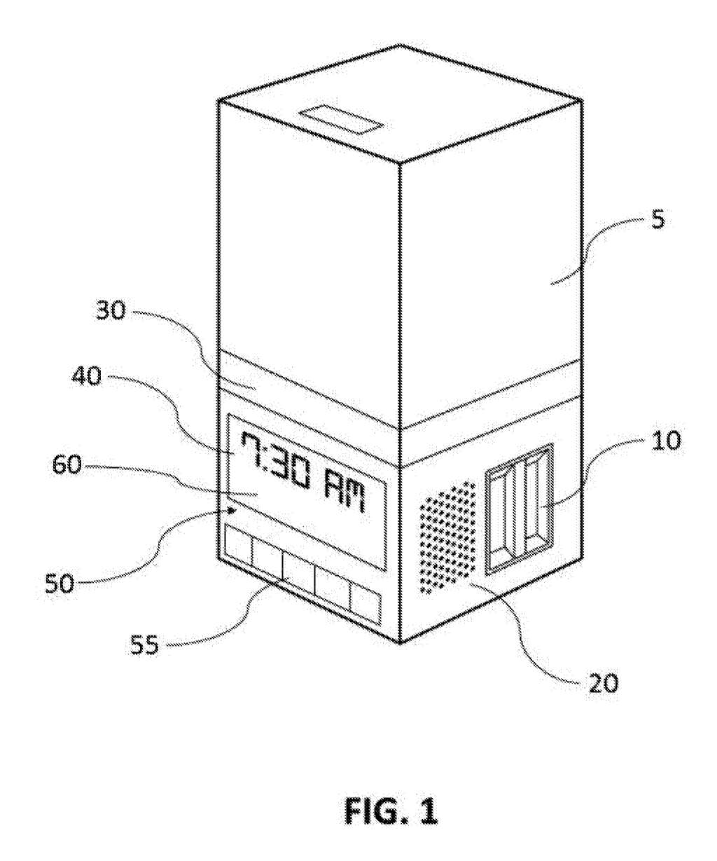

FIG. 1 shows an embodiment of a sleep and wake environment apparatus 1. The sleep and wake environment apparatus 1 incorporates several elements comprising a humidifier with humidistat 5, an aromatherapy diffuser 10, a speaker 20, a light source 30 capable of producing multiple wavelengths of the light spectrum, a clock 40, and a user interface 50 with controls 55 and a display 60. The humidifier with humidistat is optional, and in an embodiment, an optional dehumidifier is also incorporated.

In an embodiment, the aromatherapy diffuser 10 may be a dual-scent diffuser capable of diffusing a first or second fragrance or a combination of the two. For example, the dual-scent diffuser disclosed in U.S. provisional application 62/339,559, filed Jun. 7, 2016, titled “Apparatus for Controlling Fragrances,” which is incorporated herein by reference, may be used.

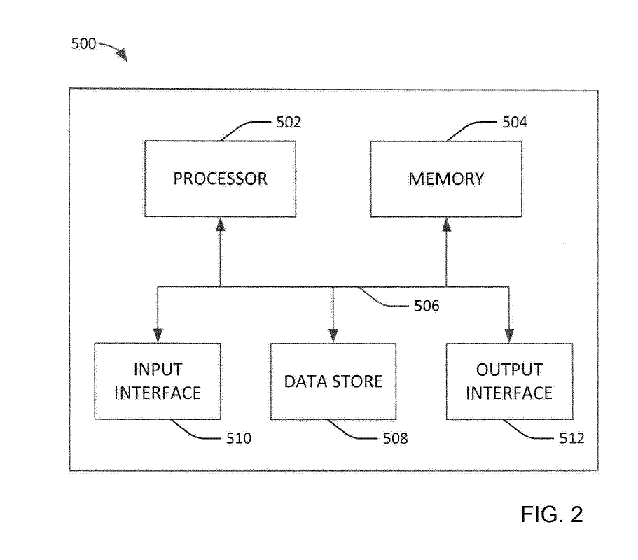

A control computing device (not shown, but housed within the sleep and wake apparatus) is in communication with the sleep and wake apparatus 1. The control computing device is configured to receive communication from the user interface 50, or a remote user interface, such as from a cellular phone or a remote computing device. The control computing device serves to control the elements of the sleep and wake apparatus 1. In an embodiment, the control computing device comprises a computing device including the computing device 500 of FIG. 2 that is in communication with a controller for each element of the sleep and wake apparatus 1.

In an embodiment, the control computing device is configured to turn on or off each element, to control volume of the speaker 20, to play sound through the speaker 20, to control the aromatherapy diffuser 10 (such as by selecting the scent or a proportional blend of two or more scents to diffuse, or by controlling a fan speed or external opening sizes of the diffuser 10), control the light source 30 (such as by controlling the intensity and/or wavelength of light), and control the optional humidifier 5 via the humidistat. The control computing device may also control and/or communicate with the alarm clock 40. In an embodiment, the control computing device is configured to control coordinated activation, deactivation, or adjustment, or any these, of the aromatherapy diffuser, the speaker, and the light source, depending on preset time intervals.

The control computing device may also communicate with the Internet and external computing devices. Through the Internet the control computing device may receive pushed updates to firmware and receive new preset programs. For example, a user may interact with a website to select and cause one or more preset programs to be sent to the sleep and wake apparatus 1. Alternatively, new or adjusted preset programs may be pushed from a remote computing device over the Internet without the consumer's request.

The clock 40 may be utilized to control timing of the wake and sleep actions. The wake actions may include combinations of sounding an alarm, diffusing one or more scents (particularly invigorating scents), emitting light of a certain wavelength and/or intensity or full-spectrum light, and activating, deactivating, and/or adjusting the optional humidifier 5 or dehumidifier (not shown).

The sleep actions include actions to be taken to aid a user in falling asleep and also actions to be performed during sleeping. The sleep actions may include combinations of playing a calming tune, song, or other noise, diffusing one or more soothing scents such as lavender, turning off or significantly lowering the light, activating, deactivating, and/or adjusting the humidifier 5.

In an embodiment, the wake and sleep actions are triggered automatically and without contemporaneous user input. By contemporaneous user input it is meant that an input for each action is required at the time the action takes place, such as pressing a button for turning on each element. Contemporaneous need not necessarily be immediate, and may also include a short delay such as 0.001 to 60, such as 1 to 10, or 2 to 5 seconds. Preset sleep and wake action programs may be stored in data storage (e.g. memory) and be triggered by a preset program, designed, for example, by doctors or sleep specialists. The sleep and wake action programs can include specific timing of sleep and wake actions. The timing may be based on specific time of day or set time intervals between actions. A single press of a preset button or another type of input from the user would thus trigger several actions in the future to be carried out by the one or more elements of the apparatus. The end-user could also access new presets via the Internet as discussed above or transfer them via electronic transmission, e.g., USB to the apparatus. In an embodiment, users could also download other users' presets as well via proprietary software. The apparatus could also be connected directly to the Internet as discussed above. Input may be received by the control computing device from the user interface 50 or over the Internet or from a remote connection such as Bluetooth, RFID, or infrared communication devices.

The sleep actions may be divided into pre-sleep and sleep actions. The sleep actions begin after the pre-sleep actions end. The pre-sleep period may last, for example, for 5 to 90, 10 to 60, or 15 to 30 minutes. Certain actions, including activations or deactivations of the elements of the apparatus discussed herein, during the pre-sleep period may gradually be adjusted, for example at a constant rate in the last 30 to 1, 15 to 5, or 10 to 3 minutes of the pre-sleep period to the levels to be performed during the sleep period.

The sleep period may last, for example, for 15 to 600 minutes, 200 to 550 minutes, or 300 to 480 minutes. In an embodiment, the duration of the sleep period is determined by the wake time set by the user as an alarm. Certain actions, including activations or deactivations of the elements of the apparatus discussed herein, during the sleep period may gradually be adjusted, for example at a constant rate in the first or last 30 to 1, 15 to 5, or 10 to 3 minutes of the pre-sleep period to the levels to be performed during the sleep period or the wake period.

The wake period may last, for example, for 5 to 60, 15 to 40, or 20 to 30 minutes and the duration may be based on when the user turns off an alarm, i.e., a user may terminate all actions with a contemporaneous input. In an embodiment, the duration of the wake period is determined by the wake time set by the user as an alarm, meaning that the wake period ends when the alarm is turned off and begins 60 to 5 minutes prior to the when alarm is set to turn on. Certain actions performed, including activations or deactivations of the elements of the apparatus discussed herein, during the wake period may gradually be adjusted, for example, at a constant rate in the first 30 to 1, 15 to 5 or 10 to 3 minutes of the wake period to the levels to be performed during the wake period.

In another embodiment, preset pre-sleep, sleep and wake action programs may be stored in data storage (e.g., memory) and may be received from internet sources or other remote computing devices or could be programmed and saved by the user as a preset and accessed by the control computing device to be executed on the apparatus's numerous elements. In an embodiment, instructions for the sleep and wake actions may be stored on a bar code read by the sleep and wake apparatus 1, on an RFID chip, or other type of data storage. For example, these may be located on a scent refill package.

The light source 30 is capable of producing multiple wavelengths of the light spectrum. In an embodiment, the light source is an incandescent or fluorescent light. The color of the emitted light may be changed by having multiple bulbs of different colors, such as by different colored coatings on the bulb, or by the use of moveable shades exterior from the bulb that can be interchanged manually or electronically. In an embodiment, the light emits only color of light. In an embodiment, the light source 30 is a fluorescent CFL full spectrum light bulb, such as those that emulate natural light. In an embodiment, the light source 30 produces a constant source of light of a single color, such as white, yellow, red, orange, blue, green, indigo, or violet, and is not a light “show.” In an embodiment, the light source 30 is exclusive of LED bulbs.

In an embodiment, a warm spectrum light is emitted in the pre-sleep. Warm spectrum light may comprise soft white light that has a yellow tint. For example, the light emitted in the pre-sleep period may have a color temperature ranging from 2000K to 3500K, such as, 2300K to 3200K, or 2500K to 3000K. In an embodiment, the light emitted in the wake period may be bright white or blue. For example, the light emitted in the wake period may have a color temperature ranging from 3500K to 7000K, such as, 4000K to 6500K, or 4500K to 6000K.

For example, a deep sleep preset could maintain a 40-60% humidity, such as 45 to 55% or 50% humidity for 2 to 4, or 2.5 to 3.5, or three hours, then adjust the humidity down to 25 to 45%, such as 30% to 40%, or 35% for waking, while varying the strength of a calming scent (lavender) from 100% to 0%, during the night, then 100% of an invigorating scent, such as peppermint 45 to 5 minutes, such as 40 to 15 minutes, or 30 minutes before wake time. The scent percent is based on the maximum throughput of air from the apparatus, which may be, for example, 25 to 1000 cfm (cubic feet per minute), such as 40 to 100 cfm, or about 50 cfm. Varying the strength from 100% to 0% may be done gradually, e.g. over an hour, or half an hour, or more abruptly such as over 15 minutes or 1 minute. Wake time may be a set time, such as 6:00 AM, or may be a predetermined amount of time after initiating the preset program.

The aromatherapy diffuser 10 in a pre-sleep period may diffuse scented oils or other aromatic material that promotes relaxation or calmness. For example, lavender, chamomile, sandalwood, ylang-ylang, vanilla, valerian, jasmine, rose, bergamot, or lilac scent may be used alone or in combination. These scents may be continued into the sleep period as well.

The aromatherapy diffuser 10 in a wake period (or just prior to a wake period) may diffuse scented oils or other aromatic material that promotes alertness and invigorates. For example, lemon, rosemary, peppermint, cinnamon, mint, or orange may be used alone or in combination.

In an embodiment, the aromatherapy diffuser has compartments for two different scents, one that is activated in the pre-sleep period and another that is activated in the wake period (or just prior to the wake period).

In an embodiment, at the beginning of the sleep period the aroma is either gradually reduced to zero or reduced to lower level such as 10% to 80%, 30% to 70%, or 50 to 65%. If aroma is diffused in the sleep period, this aroma may be gradually reduced just prior to the wake period. The wake aroma may begin immediately at the beginning of the wake period or it may not occur for several minutes into the wake period.

In an embodiment, the control computing device communicates with the speaker 20 to play various soothing/relaxing sounds including white noise, pink noise, nature sounds, or music, reading of a textual passage. In addition, to account for loud noises that may disrupt the sleep environment, the control computing device communicates with the speaker 20 to play white noise, pink noise, a specific noise cancelling algorithm, or adaptive noise cancellation algorithms. Such sounds may be activated to play during the pre-sleep and/or sleep periods.

Pink noise as mentioned in the tables is random noise having equal energy per octave, and so having more low-frequency components than white noise. Pink noise may be defined more particularly as any noise with a power spectral density of the following form

$$S ( f ) ∝ \frac{1}{{f}^{α}} ,$$

where f is frequency, and 0<α<2, wherein the exponent α is about 1, such as 1 plus or minus 0.01. In contrast, white noise is a random signal having equal intensity at different frequencies, giving it a constant power spectral density.

In an embodiment, at the beginning of the sleep period the sound is either gradually reduced to zero or reduced to lower level such as 10% to 80%, 30% to 70%, or 50 to 65%. If sound is playing in the sleep period, this noise may be gradually reduced just prior to the alarm sounding.

In the wake period, the control computing device communicates with the speaker 20 to play an alarm sound. The alarm may begin immediately at the beginning of the wake period or it may not occur for several minutes into the wake period, for example, the wake aroma or light may begin at the beginning of the wake period. The alarm may be an abrupt alarming sound, nature sounds, a musical tune, or a song. In an embodiment, the sleep sound gradually decreases to zero and then the alarm sound gradually increases in volume over 0.5 to 15 minutes, such as 1 to 10 minutes, or 2 to 5 minutes until a maximum volume is reached.

Further example preset settings are provided in Tables 1 to 5.

TABLE 1

| Preset | Description | Sequence | Aroma | Sound | Light |
|---|---|---|---|---|---|
| Sleep Well | This preset | Pre- | Sleep aroma | Pink noise at user- | Warm spectrum |
| | includes the | Sleep | 100% for 20 | specified starting | light 100% for |
| | recommended | | minutes with a | volume for 30 | 20 minutes with |
| | routine for a | | gradual reduction | minutes. | a gradual |
| | typical good | | to 0% within the | | reduction to 0% |
| | night's sleep. | | following 5 | | within the |
| | | | minutes. | | following 5 |
| | | | | | minutes. |
| | | Sleep | No aroma | After pre-sleep | No light |
| | | | | sequence, pink noise | |
| | | | | gradually reaches | |
| | | | | 60% of starting | |
| | | | | volume and | |
| | | | | maintains until wake | |
| | | | | sequence. | |
| | | Wake | Wake aroma | Pink noise gradually | No light |
| | | | gradually | reduces to 0%, | |
| | | | increases to | Sequence begins 35 | |
| | | | 100%. Sequence | minutes before wake | |
| | | | begins 25 minutes | time and ends at | |
| | | | before wake time | wake time. User- | |
| | | | and ends when | specified alarm | |
| | | | user turns it off. | gradually increases | |
| | | | | from 0% to 100%. | |
| | | | | Sequence begins 30 | |
| | | | | minutes before wake | |
| | | | | time and reaches | |
| | | | | maximum volume at | |
| | | | | wake time until user | |
| | | | | turns it off. | |

TABLE 2

| Preset | Description | Sequence | Aroma | Sound | Light |
|---|---|---|---|---|---|
| Early Morning | This preset has | Pre- | Sleep aroma | Pink noise at user- | Warm spectrum |
| | a shortened | Sleep | 100% for 15 | specified starting | light 100% for |
| | pre-sleep and | | minutes with a | volume for 20 | 15 minutes with |
| | wake sequence | | gradual reduction | minutes. | a gradual |
| | in order to | | to 0% within the | | reduction to 0% |
| | maximize | | following 5 | | within the |
| | sleep time. It | | minutes. | | following 5 |
| | would be used | | | | minutes. |
| | by someone | Sleep | No aroma | After pre-sleep | No light |
| | who is getting | | | sequence, Pink Noise | |
| | to bed late and | | | gradually reaches | |
| | rising early. | | | 60% of starting | |
| | | | | volume and | |
| | | | | maintains until wake | |
| | | | | sequence. | |
| | | Wake | Wake aroma | Pink noise gradually | No light |
| | | | gradually | reduces to 0%, | |
| | | | increases to 0% | Sequence begins 10 | |
| | | | to 100%. | minutes before wake | |
| | | | Sequence begins | time and ends 5 | |
| | | | 10 minutes before | minutes before wake | |
| | | | wake time and | time. User-specified | |
| | | | ends when user | alarm gradually | |
| | | | turns it off. | increases from 0% to | |
| | | | | 100%. Sequence | |
| | | | | begins 10 minutes | |
| | | | | before wake time and | |
| | | | | reaches maximum | |
| | | | | volume at wake time | |
| | | | | until user turns it off. | |

TABLE 3

| Preset | Description | Sequence | Aroma | Sound | Light |
|---|---|---|---|---|---|
| Noisy Room | This preset is | Pre- | Sleep aroma | Pink noise at user- | Warm spectrum |
| | designed to | Sleep | 100% for 25 | specified starting | light 100% for |
| | mask | | minutes with a | volume. | 25 minutes with |
| | environmental | | gradual reduction | | a gradual |
| | noise. | | to 0% within the | | reduction to 0% |
| | | | following 5 | | within the |
| | | | minutes. | | following 5 |
| | | | | | minutes. |
| | | Sleep | No aroma | Pink noise at user- | No light |
| | | | | specified starting | |
| | | | | volume. | |
| | | Wake | Wake aroma | Pink noise gradually | No light |
| | | | gradually | reduces to 0%, | |
| | | | increases from | sequence begins 35 | |
| | | | 0% to 100%. | minutes before wake | |
| | | | Sequence begins | time and ends at | |
| | | | 25 minutes before | wake time. User- | |
| | | | wake time and | specified alarm | |
| | | | ends when user | gradually increases | |
| | | | turns it off. | from 0% to 100%. | |
| | | | | Sequence begins 30 | |
| | | | | minutes before wake | |
| | | | | time and reaches | |
| | | | | maximum volume at | |
| | | | | wake time until user | |
| | | | | turns it off. | |

TABLE 4

| Preset | Description | Sequence | Aroma | Sound | Light |
|---|---|---|---|---|---|
| Sixty Minute | This preset | Pre-Sleep | Sleep aroma | Pink noise at user- | Warm spectrum |
| Power Nap | reduces the | (10 | 100% for 5 | specified starting | light 100% for 5 |
| | three | minutes) | minutes with a | volume. | minutes with a |
| | sequences to | | gradual reduction | | gradual |
| | occur within | | to 0% within the | | reduction to 0% |
| | the duration of | | following 3 | | within the |
| | the nap. | | minutes. | | following 5 |
| | | | | | minutes. |
| | | Sleep | no aroma | Pink noise at user- | no light |
| | | (50 | | specified starting | |
| | | minutes) | | volume. | |
| | | Wake | Wake aroma at | Pink noise stops at | no light |
| | | (determined | 100%. Sequence | wake time. User- | |
| | | by user) | begins at wake | specified alarm sound | |
| | | | time and ends | at 100% at wake time | |
| | | | when user turns it | until user turns it off. | |
| | | | off. | | |

TABLE 5

| Preset | Description | Sequence | Aroma | Sound | Light |
|---|---|---|---|---|---|
| Stressful Day | This preset | Pre- | Sleep aroma | Relaxation sound at | Warm spectrum |
| | lengthens the | Sleep | 100% for 45 | user-specified | light 100% for |
| | pre-sleep | | minutes with a | starting volume for | 45 minutes with |
| | sequence. This | | gradual reduction | 45 minutes. | a gradual |
| | preset would | | to 0% within the | | reduction to 0% |
| | be used by | | following 15 | | within the |
| | someone | | minutes. | | following 15 |
| | needing extra | | | | minutes. |
| | time to wind | Sleep | no aroma | During sleep | no light |
| | down. | | | sequence, pink noise | |
| | | | | gradually reaches | |
| | | | | 60% of starting | |
| | | | | volume and | |
| | | | | maintains until wake | |
| | | | | sequence. Sequence | |
| | | | | begins 30 minutes | |
| | | | | into pre-sleep | |
| | | | | sequence. | |
| | | Wake | Wake aroma | Pink noise gradually | no light |
| | | | gradually | reduces to 0%, | |
| | | | increases to 100%. | sequence beings 35 | |
| | | | Sequence begins | minutes before wake | |
| | | | 25 minutes before | time and ends at | |
| | | | wake time and | wake time. User- | |
| | | | ends when user | specified alarm | |
| | | | turns it off. | gradually increases | |
| | | | | from 0% to 100%. | |
| | | | | Sequence begins 30 | |
| | | | | minutes before wake | |
| | | | | time and reaches | |
| | | | | maximum volume at | |
| | | | | wake time until user | |
| | | | | turns it off. | |

The humidity component is optional in all embodiments mentioned in the Tables, and if used may be set in all time periods to be 40% to 50% humidity, such as 42% to 48% humidity. In an embodiment, the humidity may be adjusted down to 35% for waking. Consistently maintaining this level of humidity in all environments and seasons could be done with an additional dehumidifier component. In an embodiment that only includes a humidifier component (and not a dehumidifier) the humidifier may be turned on by the present program when the humidity level falls below 40%, and runs until humidity level reaches 50%.

Referring now to FIG. 2, an illustration of an exemplary computing device 500 is provided that can be used in accordance with the systems and methodologies disclosed herein. For example, the exemplary computing device 500 is a component of the controller computing device referred to above.

The computing device 500 includes data storage 508 that is accessible by the processor 502 by way of the system bus 506. The data storage 508 may include executable instructions to operate the processor 502 and other components. The computing device 500 also includes an input interface 510 that allows external devices to communicate with the computing device 500. For instance, the input interface 510 may be used to receive instructions from an external computer device or from the user interface 50 described above. The computing device 500 also includes an output interface 512 that interfaces the computing device 500 with one or more external devices. For example, the computing device 500 may display text, images, etc. by way of the output interface 512, (which may be the display screen 60 shown in FIG. 1).

It is contemplated that the external devices that communicate with the computing device 500 via the input interface 510 and the output interface 512 (which may be the user interface 50 with controls 55 and display 60) can be included in an environment that provides substantially any type of user interface with which a user can interact. Examples of user interface types include graphical user interfaces, natural user interfaces, and so forth. For instance, a graphical user interface may accept input from a user employing input device(s) such as a keyboard, mouse, remote control, or the like and provide output on an output device such as a display. Further, a natural user interface may enable a user to interact with the computing device 500 in a manner free from constraints imposed by input device such as keyboards, mice, remote controls, and the like. Rather, a natural user interface can rely on speech recognition, touch and stylus recognition, gesture recognition both on screen and adjacent to the screen, air gestures, head and eye tracking, voice and speech, vision, touch, gestures, machine intelligence, and so forth.

Additionally, while illustrated as a single system, it is to be understood that the computing device 500 may be a distributed system. Thus, for instance, several devices may be in communication by way of a network connection and may collectively perform tasks described as being performed by the computing device 500.

Various functions described herein can be implemented in hardware, software, or any combination thereof. If implemented in software, the functions can be stored on or transmitted over as one or more instructions or code on a computer-readable medium. Computer-readable media includes computer-readable storage media. A computer-readable storage media can be any available storage media that can be accessed by a computer. By way of example, and not limitation, such computer-readable storage media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, include compact disc (CD), laser disc, optical disc, digital versatile disc (DVD), floppy disk, and BLU-RAY (BD), where disks usually reproduce data magnetically and discs usually reproduce data optically with lasers. Further, in an example, a propagated signal is not included within the scope of computer-readable storage media or display data. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio and microwave are included in the definition of communication medium. Combinations of the above should also be included within the scope of computer-readable media.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

It is claimed:

1. A sleep and wake environment apparatus comprising:
an aromatherapy diffuser;
a speaker;
a light source;
a clock;
a user interface with controls and a display, each of which are in communication with a control computing device configured to control the aromatherapy diffuser, the speaker, the light source, and the clock automatically and without contemporaneous user input.

2. The sleep and wake environment apparatus of claim 1, wherein each of the aromatherapy diffuser, the speaker, the light source, the clock, and the user interface with controls and a display are contained within a single housing.

3. The sleep and wake environment apparatus of claim 1, wherein the control computing device is configured to receive communication from a remote user interface.

4. The sleep and wake environment apparatus of claim 1, wherein the control computing device is configured to control coordinated activation, deactivation, or adjustment, or any these, of the aromatherapy diffuser, the speaker, and the light source, depending on preset time intervals.

5. The sleep and wake environment apparatus of claim 4, wherein the preset time intervals correspond to at least two of pre-sleep, sleep, or wake time periods.

6. The sleep and wake environment apparatus of claim 5, wherein in the pre-sleep time period at least two of the aromatherapy diffuser, the speaker, or the light source are activated.

7. The sleep and wake environment apparatus of claim 5, wherein in the sleep time period at least one of the aromatherapy diffuser, the speaker, or the light source are activated, deactivated, or adjusted automatically and without contemporaneous user input.

8. The sleep and wake environment apparatus of claim 5, wherein in the wake time period at least two of the aromatherapy diffuser, the speaker, or the light source are activated, deactivated, or adjusted automatically and without contemporaneous user input.

9. The sleep and wake environment apparatus of claim 5, wherein in the pre-sleep time period at least two of the aromatherapy diffuser, the speaker, or the light source are activated;
wherein in the sleep time period at least one of the aromatherapy diffuser, the speaker, or the light source are activated, deactivated, or adjusted automatically and without contemporaneous user input;
wherein in the wake time period at least two of the aromatherapy diffuser, the speaker, or the light source are activated, deactivated, or adjusted automatically and without contemporaneous user input.

10. The sleep and wake environment apparatus of claim 1, wherein the light source is capable of producing multiple wavelengths of the light spectrum.

11. The sleep and wake environment apparatus of claim 1, further comprising a humidifier wherein the control computing device is configured to control the humidifier automatically and without contemporaneous user input.

12. The sleep and wake environment apparatus of claim 4, wherein at least one of the activation, deactivation, or adjusting are gradually performed in a time period of 30 minutes to 1 minute.

13. A method of controlling a sleep and wake environment, in a computer controlled apparatus, the method comprising:
automatically and without contemporaneous user input:
activating in a pre-sleep time period at least two of an aromatherapy diffuser, a speaker, or a light source;
activating, deactivating, or adjusting in a sleep time period at least one of the aromatherapy diffuser, the speaker, or the light source;
activating, deactivating, or adjusting in a wake time period at least two of the aromatherapy diffuser, the speaker, or the light source.

14. The method of claim 13, wherein in the pre-sleep time period, activating the light source, activating the aromatherapy diffuser to emit a relaxing scent, and activating the speaker to emit a calming tune, song, or other noise.

15. The method of claim 13, wherein in the sleep time period, deactivating the light source, deactivating or adjusting the aromatherapy diffuser, and deactivating the speaker.

16. The method of claim 13, wherein in the wake time period, activating the aromatherapy diffuser to emit an invigorating scent and activating the speaker.

17. The method of claim 13, wherein in the pre-sleep time period, activating the light source, activating the aromatherapy diffuser to emit a relaxing scent, and activating the speaker to emit a calming tune, song, or other noise;
in the sleep time period, deactivating the light source, deactivating or adjusting the aromatherapy diffuser, and deactivating the speaker; and
in the wake time period, activating the aromatherapy diffuser to emit an invigorating scent and activating the speaker.

18. The method of claim 13, wherein at least one of the activating, deactivating, or adjusting of the light source, the aromatherapy diffuser, and the speaker are gradually performed in a time period of 30 minutes to 1 minute.

19. The method of claim 13, wherein the computer controlled apparatus communicates through the Internet to acquire and store instructions to control the activating, deactivating or adjusting of the light source, the aromatherapy diffuser, and the speaker.

20. The method of claim 13, further comprising activating in a pre-sleep time period a humidifier.
